(19) United States
(12) Patent Application Publication
(10) Pub. No.: US 2012/0159732 A1
(43) Pub. Date: Jun. 28, 2012

(54) Back tanning belt

(76) Inventor: Bruce Killion, Venice, FL (US)

(21) Appl. No.: 13/374,224

(22) Filed: Dec. 16, 2011

Related U.S. Application Data
(60) Provisional application No. 61/459,809, filed on Dec. 20, 2010.

(51) Int. Cl. A61M 35/00

(52) U.S. Cl. 15/210.1

(57) ABSTRACT

A method and belt-like device is described for applying commercially-available beneficial liquids or semi-solids such as a tanning or sunblock lotion, insect repellentor therapeutic formulation to the back and other hard-to-reach skin areas of the human body by the person desiring to apply the same acting alone without the assistance of another person. The device has a removable absorbent pad to hold multiple loadings of the liquid or semi-solid and release it as it dragged horizontally or diagonally back and forth over the user's desired skin area by the user's hand and arm motion.

### BACKGROUND OF THE INVENTION

The method and device is a new invention to solve an old problem: applying a commercially-available beneficial liquid or semi-solid such as a tanning or sunblock lotion, insect repellent or a therapeutic formulation to a person's back and other hard-to-reach skin areas of the body when the person is alone. Even if a person could reach the desired area with his or her right or left hand, applying the liquid or semi-solid fully, evenly and in uniform thickness onto the desired skin area is almost impossible.

### FIELD AND SUMMARY OF THE INVENTION

The present document relates to the field of personal care and human healthcare. In particular, this document describes a method and belt-like device with a removable absorbent pad which enables a person who is alone to self-apply commercially-available liquids or semi-solids evenly to the back and other hard-to-reach skin areas of his or her body for the achieving certain desired cosmetic, preventative or therapeutic purposes.

### DETAILED DESCRIPTION OF THE INVENTION

The device is belt-like and is several feet long and several inches in width and depth. The device comprises an application pad made of a material which is capable of absorbing, holding and releasing a desired liquid or semi-solid supported by a soft fabric, each end of which has a handle attached. In one embodiment, the pad is constructed of foam rubber, and the supporting fabric is woven cotton. The pad is removable and can be washed after each use of the device. The pad is attached to the supporting fabric and held in place by Velcro strips attached to the belt as shown in the drawings.

By impregnating the belt-like device with a desired liquid or semi-solid and then moving the device back and forth in horizontal or diagonal dragging motion over the body area or areas to receive the liquid or semi-solid by means of two long cloth handles the ends of which are held in each hand, a person without the assistant of another person can self-apply the liquid or semi-solid to the hard-to reach skin area desired to receive the liquid or semi-solid, and can apply the liquid or semi-solid fully, evenly and in uniform thickness for maximizing the benefit thereof.

In one embodiment, the liquid or semi-solid is a commercially-available skin tanning or sunblock lotion to give the skin a desired appearance and to protect the skin from harmful solar radiation or radiation from an artificial light source.

In another embodiment, the liquid or semi-solid is a commercially-available insect repellent.

In another embodiment, the liquid or semi-solid is a commercially-available formulation having therapeutic properties, which formulation is either available to the user of the device over-the-counter or by prescription.

In one embodiment, the absorbent pad is loaded multiple times by the user of the device with commercially-available liquid or semi-solid products by pouring the same on the pad.

In another embodiment, the absorbent pad is loaded a single time by the seller of the device with one of its own liquid or semi-solid products.

### DESCRIPTION OF THE DRAWINGS

FIG. 1. A front view of the device indicated by a white background including a view of the attached application pad indicated by wide-angled hatch marks and the Velcro strips indicated by the darker, close-angled hatch marks. The measurements are included as one embodiment of the invention.

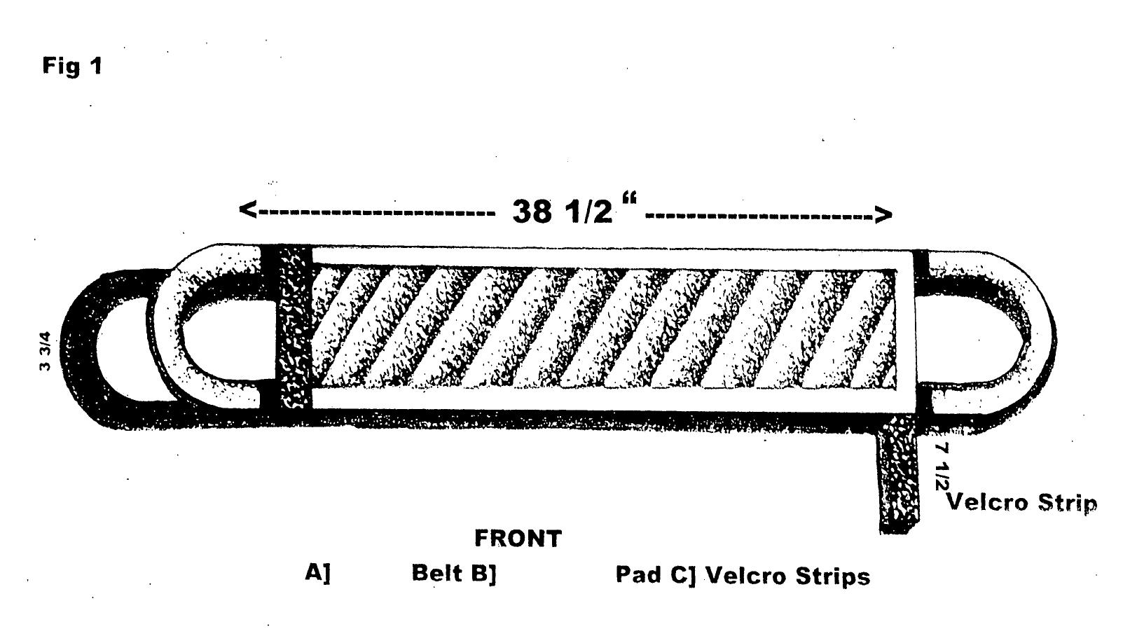

FIG. 2. A partial front and side view of the device. The measurements are included as one embodiment of the invention.

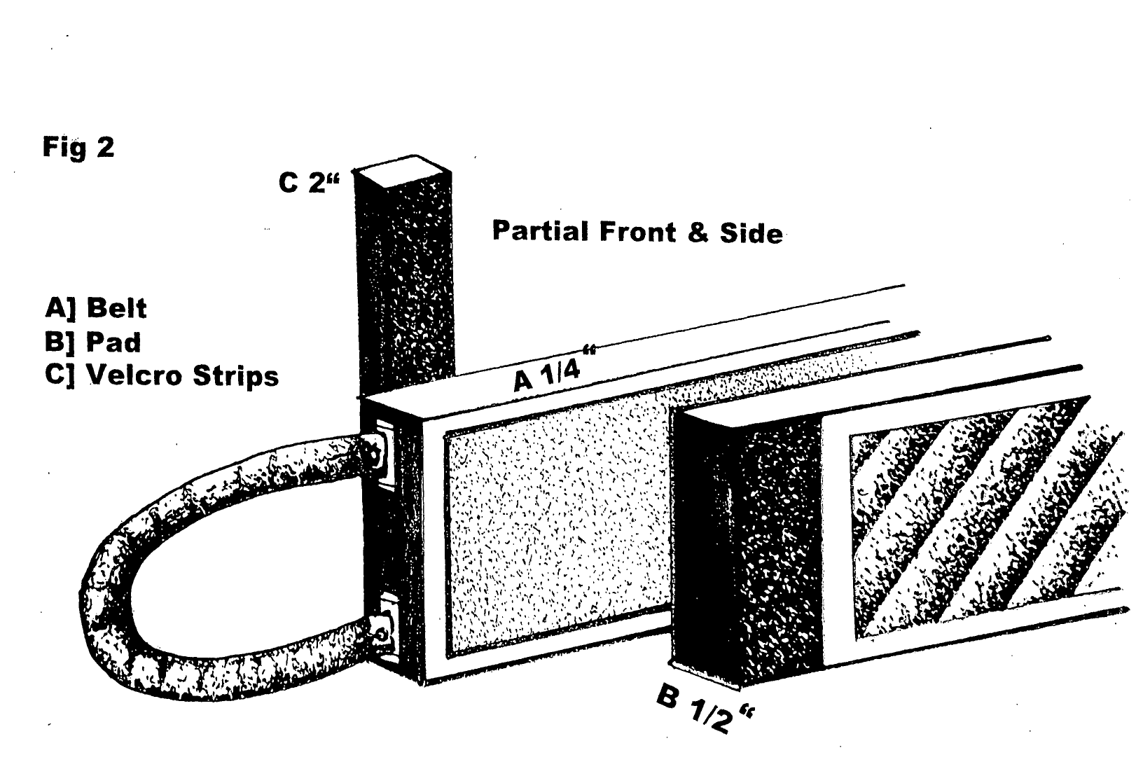

FIG. 3. A side view of the device, including the handle detail. The measurements are included as one embodiment of the invention.

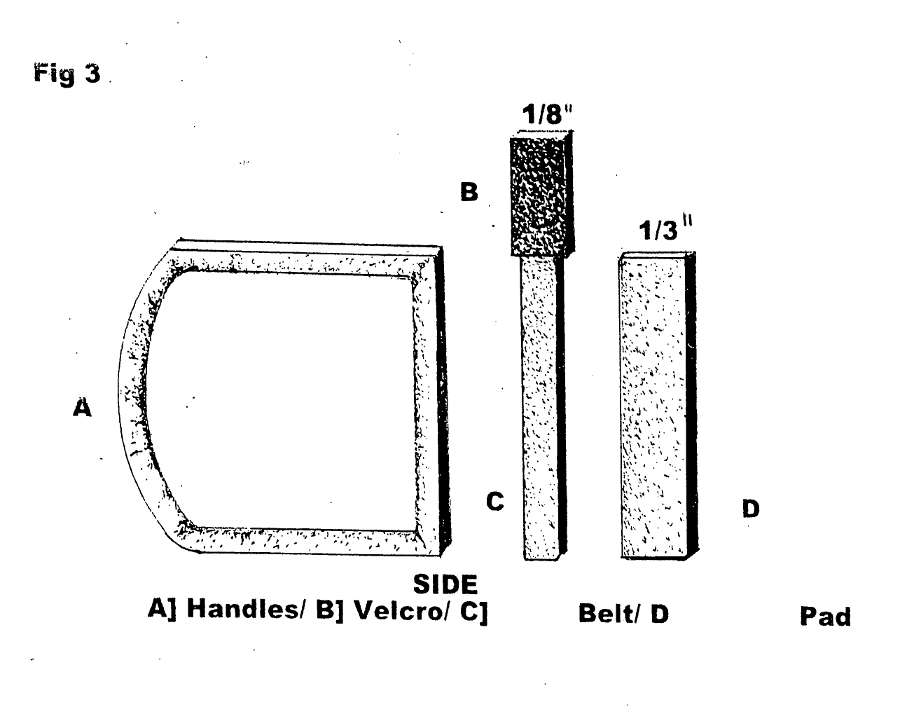

The claimed invention is:

1. A device comprising a soft, washable fabric supporting a pad enabling a user to self-apply commercially-available liquids or semi-solids absorbed into the pad to the surface of the skin on impossible- or difficult-to-reach areas of the body such as the back, shoulder blades, back of the neck, buttocks and or back of the legs which is manipulated by the coordinated motion of both hands and arms of the user, allowing the user without assistance of another person to apply such liquids or semi-solids fully, evenly and in uniform thickness on such areas of the user's skin surface for the purpose of such skin areas receiving the benefits of the applied liquid or semi-solid.

2. The device of claim 1 wherein such pad is made from a synthetic porous material which absorbs such liquids or semi-solids applied to the pad and releases the same when the pad is applied by the user to the skin of such body areas.

3. The device of claim 1 wherein such pad is made from a natural porous material which absorbs such liquids or semi-solids applied to the pad and releases the same when the pad is applied by the user to the skin of such body areas.

4. The device of claims 1, 2 and 3 wherein such pad is removable by the user from the supporting fabric.

5. The device of claims 1, 2, 3 and 4 wherein such pad may be removed by the user from the supporting fabric for the purpose of the user's replacing the removed pad with a new pad of the same porous material or of a different porous material impregnated by a commercial entity with such a liquid or semi-solid.

6. The device of claims 1, 2, 3 and 4 wherein such pad may be removed by the user from the supporting fabric for the purpose of the user's replacing the removed pad with a new pad of the same porous material or of a different porous material which does not contain any such liquids or semi-solids.

7. The device of claims 1, 2, 3 and 4 wherein such pad is capable of being be cleaned.

8. The device of claims 1, 2, 3, 4 and 7 wherein such pad may be removed by the user from the supporting fabric for the purpose of the user's applying such liquids or semi-solids to the removed pad.

9. The device of claims 1, 2, 3 wherein such pad is not removable and is capable of being cleaned by hand or machine.
